(19) United States
(12) Patent Application Publication
(10) Pub. No.: US 2006/0029979 A1
(43) Pub. Date: Feb. 9, 2006

(54) Electrochemical luminescence composite material with anti-biofouling properties

(75) Inventors: Chunxue Bai, Shanghai (CN); Wei Zhong, Shanghai (CN); Yuanlin Song, Shanghai (CN)

Correspondence Address: CROWELL & MORING LLP;INTELLECTUAL PROPERTY GROUP, P.O. BOX 14300, WASHINGTON, DC, 20044-4300, US

(73) Assignee: Fudan University, Shanghai (CN)

(21) Appl. No.: 11/196,305

(22) Filed: Aug. 4, 2005

(30) Foreign Application Priority Data
Aug. 5, 2004 (CN) 200410053454.1

(51) Int. Cl. G01N 33/53; C12M 1/34

(52) U.S. Cl. 435/7.1; 435/287.2

(57) ABSTRACT

The present invention relates to the preparation ad application of a high-sensitive electrochemical luminescent composite material which has anti-biofouling properties useful as a sensor material. This material is prepared by immobilization of electrochemical luminescent material into polymer containing phospholipid groups, wherein, the electrochemical luminescent material including ruthenium complex, osmium complex, etc.; the phospholipid containing polymer is the copolymer of 2-methacryloyloxyethyl phosphorylcholine (MPC) and other polymerisable monomers. Animal experiment results revealed that this composite material has good anti-biofouling properties; it can be used in producing various sensors for bio-related detections.

### FIELD OF INVENTION

The present invention relates to the preparation and application of a high-sensitive electrochemical luminescent composite material which has anti-biofouling properties useful as a sensor material.

### TECHNICAL BACKGROUND

Electrochemical luminescence is the luminescence exited by electrochemical reactions. It is highly sensitive, so it has been used in the detection of many substances. If those high sensitive electrochemical luminescent materials could be fixed onto the distal surface of a detector or an optical fiber, a lot of expensive indicator materials might be saved, the instrument structure and the operation might also be simplified. This can contribute to the extension of the application area of the detection method and the corresponding instruments.

There are many kinds of electrochemical luminescent materials, among those, the most frequently reported is ruthenium complexes, such as ruthenium(I tis(bipyridine) complex and its derivatives. There have been ample of reports about the immobilization of ruthenium(II) tris(bipyridine) complexes, for example, make Langmuir-Blodgett film or self-assembled films from ruthenium(II) tris(bipyridine) complexes and its derivatives, or fix them into cationic ion-exchange membrane. But the stability of those immobilized luminescent materials is not good enough; it may be washed away when put into the solution. O. Dvorak and M. K. De Armond (J. Phys. Chem. 1993, 97: 2646) first report the immobilization of ruthenium (II) tris(bipyridine) complex by sol-gel method. A. N. Khramov et al (Anal. Chem. 2000, 72: 32943) immobilize ruthenium(II) trio(bipyridine) complex into Nafion-silica composite film by ion-exchanging method to prepare a modified electrode with much more improved sensitivity and stability. However, it still had a lack of a long-term stability.

Instruments employing above mentioned principle have already been commercialized, for example, high sensitive oxygen sensor, made by coating the distal of optical fiber with fluorescence-quenching ruthenium complexes, has already been used in the detection and research in outer space, as well as the environmental and soil monitoring. Compared with conventional instrument with the same function, it has the advantage like compact size, long service life, wide measurement range, rapid response, good repeatability, stable performance as well as the possibility for in-situ detection.

With the development of modern medicine, there are more and more requirements for the real-time measurements of many parameters of human body, such as the concentration of oxygen and some ions in blood as well as the pH of blood, especially in the first aid of patients with critical ill. For most of the clinically used instruments, here are dysfunction problems of the sensor after contacting with blood for certain time, caused by the adhesion of proteins such as platelets onto the surface of sensor. For the sensors used in other application area such as bio-reactors, there are also similar bio-fouling problems.

Phospholipid such as phosphorylcholine is the main component of the outer surface of biofilms. As a polar molecule, berg both positive and negative charge, it's an electrically Hal molecule as a whole. It is strongly hydrophilic, can prevent the reversible adhesion of proteins on its surface. Polymers bearing phosphorylcholine groups have already been applied on the surface of the biomedical materials and devices. They can reduce the foreign body reaction when in contact with body fluid such as blood, tear or urine. Most of the reported phosphorylcholine containing polymer are the copolymers of 2-methacryloyloxyethyl phosphorylcholine (MPC) and other monomers, MPC copolymers have been used on blood-contacting medical devices like coronary stents, catheters and blood dialysis membranes, etc., for the improvement of the hemocompatibility of the devices, i.e., to reduce the adhesion of the proteins in blood as well as the chance of thrombosis. It has also been reported as a surface protein-resist coating of the sensitive layer of fluorescent sensors. However, there is no report on the application of this kind of polymer on the implantable chemo-luminescent composite materials or sensors made from such composite materials.

### DETAILED DESCRIPTION OF THE INVENTION

The aim of the present invention is to propose a new electro-chemical luminescent composite material, which combines the high biocompatibility as well as anti-protein-adhesion property of phosphorylcholine polymers and the high sensitivity of the chemo-luminescent materials; and the way it is prepared, as well as its application as a sensor material.

The electro-chemical luminescent composite materials of the present invention are prepared by immobilization of electro-chemical luminescent into polymers containing phosphorylcholine groups. The content of the electro-chemical luminescent material in the composite material maybe in the range of 0.05-50% by weight, and the rest are polymers.

The electro-chemical luminescent materials in the present invention are materials which can be dissolved in certain organic solvents, including ruthenium complexes, osmium complexes, plumbum complexes, platinum and palladium complexes, porphyrin derivatives, rhenium complexes, transition metal porphyrin complexes, maybe one of these materials or mixture of more than one of them, or the mixtures of these materials with other materials like silica sol.

The phosphorylcholine-containing polymers in the present invention are copolymers of 2-methacryloyloxyethyl phosphorylcholine PC) and other polymerisable monomers. These copolymers can be obtained by free radical copolymerization of MPC with one or more than one monomers from the following monomers: (methyl, ethyl, propyl, butyl, amyl, hexyl, heptyl, octyl, nonyl, decyl, dodecyl, hexadecyl, octadecyl)acrylate or methacrylate; hydroxyethyl acrylate or methacrylate; hydroxypropyl acrylate or methacrylate; ethylene glycol acrylate or methacrylate; ethylene glycol methyl ether acrylate or methacrylate; poly(ethylene glycol)acrylate or methacrylate; poly(ethylene glycol)methyl ether acrylate or methacrylate; N-vinyl pyrrolidone; vinyl acetate; double-bond-containing silane coupling agent, such as: γ-methacryloxypropyl trimethoxysilane, γ-methacryloxypropyl triethoxysilane, vinyltris(2-methoxyethoxy)silane, methyltrivinylmethyl diethoxysilane, etc.

The preparation procedure of the electro-chemical luminescent composite materials in the present invention is as follows; (1) dissolve the electro-chemical luminescent materials in their correspondent solvent; (2) mix together with the solution of phosphorylcholine-containing polymer; (3) eliminate the solvents in the above mentioned mixed solution. The electro-chemical luminescent composite material with anti-biofouling property is thus obtained.

The electro-chemical luminescent composite materials in the present invention have good anti-biofouling efficiency; they can be used to produce various anti-biofouling biosensors.

For example, make electro-chemical luminescent composite material into film, adhere the film to the distal end of an optical fiber; or directly coat the end face of the optical fiber with the solution of electro-chemical luminescent composite material, then dry the optical fiber to remove the solvent, the optical fiber sensor with anti-biofouling property is thus obtained.

For instance, a ruthenium complex composite material prepared according to example 1 in the present invention showed markedly sensitivity and repeatability to oxygen partial pressure. It can be found from FIG. 1 that this complex material reacted sensitively, and rapidly (reach equilibrium at less than 1 minute) to oxygen partial pressure. A compact sized fluorescent instrument for the continuous and in-situ measurement of blood oxygen partial pressure can be assembled with the light source, fluorescent filter, photoelectric cell, as well as the sensor produced by coating the end face of the optical fiber with this composite material.

### BRIEF DESCRIPTION OF DRAWING

FIG. 1 is a graph showing the sensitivity to oxygen partial pressure of a chemo-luminescent composite material produced according to example 1.

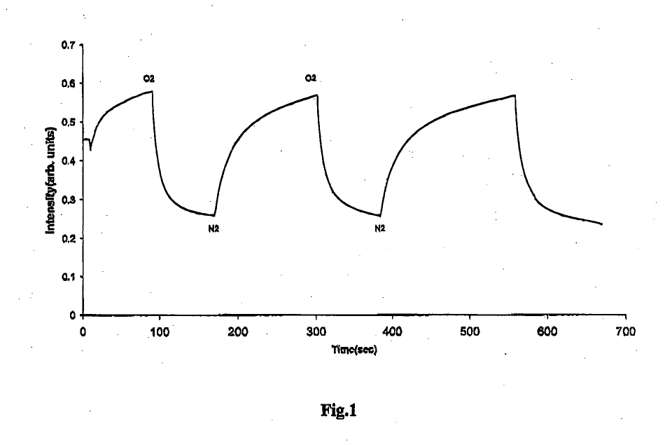

### EXAMPLES

### Example 1

First, dissolve the following momomers: 30 grams of MPC, 68 grams of butyl methacrylate, 2 grams of γ-methacryloxypropyl triethoxysilane and 0.1 gram of azobisisobutyronitrile (AIBN) as initiator into 200 ml ethanol. Bubble the solution with argon for 1 hour to eliminate oxygen. Then heat the solution to 70° C. with a thermostated bath, react under magnetic stirring for 24 hours. After that, cool the solution to room temperature, precipitate in an excess amount of hexane. After drying, the precipitate is dissolve in ethanol and precipitate in hexane again. The final precipitate is collected and dried in vacuum for 24 hours at room temperature. 90 grams of phosphorylcholine containing polymer can be thus obtained.

Dissolve 0.1 gram of a hydrophobic ruthenium complex, tris(4,7-diphenyl-1,10-phenanthroline)-ruthenium (II) bis(hexafluorophosphate) into 10 ml methane, put 0.8 gram of the above mentioned phosphorylcholine containing polymer into the same solution, magnetically stir the solution until both are dissolved, 20 μl of water was added to the filtered solution, and mixed with stirring until uniform. One distal end of an optical fiber is coated with the obtained solution, and is dried in oven for 5 hours at 70° C. An anti-biofouling optical fiber based oxygen sensor is thus obtained. It has rapid response and good repeatability, and can be used continuously under bio-fouling environment,

### Example 2

First, dissolve the following momomers: 15 grams of MPC, 10 grams of poly(ethylene glycol)methyl ether methacrylate (M=360), 10 grams of ethylene glycol methyl ether methacrylate, 63 grams of dodecyl methacrylate, 2 grams of γ-methacryloxypropyl trimethoxysilane and 0.1 gram of AIBN as initiator into ethanol/THF mixed solvent (50/50, v/v). Bubble the solution with argon for 1 hour to eliminate oxygen. Then heat the solution to 70° C. with a thermostated bath, react under magnetic sting for 24 hours. After that, cool the solution to room temperature, precipitate in an excess amount of hexane. After drying, the precipitate is dissolve in ethanol/THF and precipitate in hexane again. The final precipitate is collected and dried in vacuum for 24 hours at room temperature, 92 grams of phosphorylcholine containing polymer can be thus obtained.

Mix together the following reagents, 1 ml tetraethoxysilane (TEOS), 0.2 ml water, 20 μl of 0.1 mol/l hydrochloric acid aqueous solution, and 1 ml ethanol. After standing for 3 hours, a silica gel is obtained. Then add 0.1 gram of the imidazophenanthroline derivative of Ru(2,2′-bipyridine)₂ Cl₂.2H₂O to the silica gel and mix until uniform.

Dissolve 0.9 gram of the phosphorylcholine containing polymer prepared in His example into 15 ml ethanol/THF mixed solvent (50/50, v/v), then mix this solution thoroughly with the silica gel obtained in this example, filtrate after standing in room temperature for 2 hours. Coat this solution onto one silanized distal end of an optical fiber, then dry the optical fiber for 5 hours in an 70° C. oven. A biofouling-resist pH sensor for blood or protein-rich solution is thus obtained.

### Example 3

First, dissolve the following momomers: 20 grams of MPC, 8 grams of N-vinyl pyrrolidone, 5 grams of β-hydroxyethyl methacrylate, 67 grams of butyl acrylate, and 0.1 gram of AIBN as initiator into ethanol/THF mixed solvent (50/50, v/v). Bubble the solution with argon for 1 hour to eliminate oxygen Then heat the solution to 75° C. with a thermostated bath, react under magnetic stirring for 24 hours. After that, cool the solution to room temperature, precipitate in an excess amount of hexane. After drying, the precipitate is dissolve in ethanol/THF and precipitate in hexane again. The final precipitate is collected and dried in vacuum for 24 hours at room temperature. 89 grams of phosphorylcholine containing polymer can be thus obtained.

Dissolve 1 gram of the phosphorylcholine containing polymer in this example in 10 ml of THF, then stir thoroughly after put 80 mg of 2,6-di-O-isobutyl-β-cyclodextrin (DOB-β-CD) and 20 mg of meso-tetra(4-methoxylphenyl)porphyrin (TMOPP) into this solution. Cast this solution onto clean and leveled glass plate, after air drying in room temperature, a clear membrane of about 5 μm thick can be obtained.

Cut a small piece of the obtained membrane, stick it to one distal end of an optical fiber using transparent cyanoacrylate glue. A biofouling resist sensor for CO₂ measurement is thus obtained. It has a response range between 4×10⁻⁷ to 4×10⁻⁵ mol/L of [H₂CO₃] in water. It has not only fist response, but good repeatability.

1. An anti-biofouling electrochemical luminescent composite material characterized in that said material comprises phospholipid polymers immobilized electrochemical luminescent materials into, wherein the content of said electro-chemical luminescent materials is between 0.05-50 wt % of the total amount of said phospholipid polymers.

2. An anti-biofouling electrochemical luminescent composite material as claimed in claim 1 wherein the electro-chemical luminescent material is selected from the group consisted of ruthenium complexes, osmium complexes, plumbum complexes, platinum and palladium complexes, porphyrin derivatives, rhenium complexes, transition metal porphyrin complexes, their mixture, and their mixtures with other materials such as silica sol.

3. An anti-biofouling electrochemical luminescent composite material as claimed in claim 1 wherein the phospholipid polymers are copolymers of 2-methacryloyloxyethyl phosphorylcholine and other polymerisable monomes.

4. An anti-biofouling electrochemical luminescent composite material as claimed in claim 3 wherein the other polymerisable monomers are selected from the group consisted of (methyl, ethyl, propyl, butyl, amyl, hexyl, heptyl, octyl, nonyl, decyl, dodecyl, hexadecyl, octadecyl)acrylate or methacrylate; hydroxyethyl acrylate or methacrylate; hydroxypropyl acrylate or methacrylate; ethylene glycol acrylate or methacrylate; ethylene glycol methyl ether acrylate or methacrylate; poly(ethylene glycol)acrylate or methacrylate; poly(ethylene glycol)methyl ether acrylate or methacrylate; N-vinyl pyrrolidone; vinyl acetate; double-bond-containing silane coupling agent, such as: γ-methacryloxypropyl trimethoxysilane, γ-methacryloxypropyl triethoxysilane, vinyltris(2-methoxyethoxy)silane, methyltrivinylmethyl diethoxysilane.

5. A method for preparing the anti-biofouling electrochemical luminescent composite material as claimed in claim 1, comprising: (1) dissolving the electro-chemical luminescent materials in their correspondent solvent; (2) mixing together with the solution of phosphorylcholine-containing polymer; (3) eliminating the solvents in the above mentioned mixed solution; the electro-chemical luminescent composite material with anti-biofouling property is thus obtained.

6. An application of the anti-biofouling electrochemical luminescent composite material as claimed in claim 1 in the field of biofouling-resistant biosensor.
